(19) United States
(12) Patent Application Publication
(10) Pub. No.: US 2007/0224141 A1
(43) Pub. Date: Sep. 27, 2007

(54) Kit containing two compositions

(75) Inventors: Agnes Themens, Bourg La Reine (FR); Nathalie Geffroy, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/527,625

(22) Filed: Sep. 27, 2006

Related U.S. Application Data
(60) Provisional application No. 60/725,305, filed on Oct. 12, 2005.

(30) Foreign Application Priority Data
Sep. 27, 2005 (FR) 05 52908

(51) Int. Cl. A61K 8/81

(52) U.S. Cl. 424/63

(57) ABSTRACT

The present invention relates to a kit containing a first and a second cosmetic composition, the first composition containing at least one silicone elastomer and concave particles of a siliconized material, notably in the form of portions of hollow spheres, the second composition containing a physiologically acceptable medium. This kit can be used to provide make-up that is uniform, and/or masks skin blemishes and/or avoids accentuating wrinkles or lines.

### REFERENCE TO PRIOR APPLICATIONS

This application claims priority to U.S. provisional application 60/725,305 filed Oct. 12, 2005, and to French patent application 0552908 filed Sep. 27, 2005, both incorporated herein by reference.

### FIELD OF THE INVENTION

The present invention relates to a kit, preferably a skin make-up kit, comprising at least two compositions capable of, and preferably intended to be, applied on one another, one of the compositions comprising a silicone elastomer and siliconized concave particles. The invention also relates to a cosmetic method of skin make-up, comprising the application of the kit compositions to the skin and to its use for make-up.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Make-up compositions, in particular foundation compositions, generally contain a pulverulent phase comprising pigments and fillers. They are used for masking imperfections of skin colour, such as red patches or discolorations. The application of these compositions often leads to marking of irregularities of relief of the skin, such as wrinkles or lines, making them more conspicuous, owing to migration of the particulate phase into the wrinkles.

Using a lower content of pulverulent material is a known means of limiting these problems of marking. In this case, the foundation compositions no longer provide effective masking of the defects of skin colour.

There is therefore a need for make-up products that effectively camouflage defects of skin relief and colour, while avoiding the marking of wrinkles or lines.

### SUMMARY OF THE INVENTION

The inventors have demonstrated that it is possible to obtain such products by using a make-up kit containing a first composition comprising a silicone elastomer and concave siliconized particles and a second composition comprising a pulverulent colourant.

Application of the first composition on the skin camouflages defects of skin colour and relief, and prevents marking of the irregularities of relief of the skin during application of the second composition, although the compositions can be applied in any order. Preferably, the compositions are applied, at least partially, one over the other.

### BRIEF DESCRIPTION OF THE DRAWING

FIG. 1 shows a concave particle of portions of spheres of bowl shape in cross-section. The width W2 corresponds to the diameter of the particles.

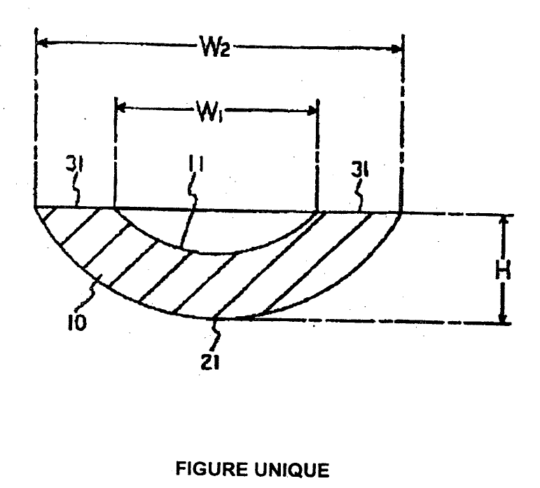

### DETAILED DESCRIPTION OF THE PREFERRED EMBODIMENTS

More precisely, the invention relates to a skin make-up kit, comprising a first and a second cosmetic composition, the first composition comprising at least one silicone elastomer and concave particles of a siliconized material, preferably in the form of portions of hollow spheres, the second composition comprising a physiologically acceptable medium. However, the kit can comprise further (e.g., third, fourth, etc. compositions).

The invention also relates, according to a second aspect, to a cosmetic (non-therapeutic) method of skin make-up, comprising the following stages:
i) forming a first deposit on a keratinic material;
ii) on all or part of the first deposit, forming a second deposit, said first and second deposits resulting from the application of the first and second compositions of the kit as described previously, where preferably the first deposit is formed of the first composition.

The invention further relates, according to a third aspect, to use of the invention kit, to obtain make-up that is uniform, and/or masks skin blemishes and/or avoids accentuating wrinkles or lines.

As used herein, a physiologically acceptable medium is a medium compatible with one or more keratinic materials, notably the skin and the lips, and in particular a cosmetic medium.

Siliconized Concave Particles

The siliconized concave particles, also termed concave particles of a siliconized material herein, contained in the first composition according to the invention are in particular particles of portions of hollow spheres comprising a siliconized material. The second composition of the kit according to the invention can also include siliconized concave particles, if desired. The particles preferably have an average diameter less than or equal to 10 μm, notably in the range from 0.05 μm to 10 μm, preferably in the range from 0.1 to 8 μm, more preferably in the range from 0.2 to 7 μm and even more preferably in the range from 0.5 to 5 μm, including 0.2, 0.4, 0.6, 0.8, 1.5, 2, 2.5, 3, 3.5, 4, 4.5, 5, 5.5, 6, 6.5, 7, 7.5, 8, 8.5, 9 and 9.5 μm and all ranges and subranges between stated values. By average diameter, we mean the largest dimension of the particle. The portions of hollow spheres used in the composition according to the invention can for example have the form of truncated hollow spheres, having a single opening communicating with their central cavity, and having a cross-section in the shape of a horseshoe or an arch.

A preferred organosilicon material is a crosslinked polysiloxane with a three-dimensional structure; it preferably comprises, or is constituted of, the units of formula (I): SiO₂ and of formula (II): R¹SiO_{1.5} in which R¹ denotes an organic group having a carbon atom joined directly to the silicon atom. The organic group can be a reactive organic group or an unreactive organic group, and preferably an unreactive organic group.

The unreactive organic group can for example be a C₁-C₄ alkyl group, notably a methyl, ethyl, propyl or butyl group, or a phenyl group, and preferably a methyl group.

The reactive organic group can for example be an epoxy group, a (meth)acryloxy group, an alkenyl group, a mercaptoalkyl group, an aminoalkyl group, a haloalkyl group, a glyceroxy group, a ureido group, or a cyano group. Preferably, the reactive organic group can be an epoxy group, a (meth)acryloxy group, an alkenyl group, a mercaptoalkyl group, or an aminoalkyl group. The reactive organic group generally contains from 2 to 6 carbon atoms, notably from 2 to 4 carbon atoms.

As epoxy group, included are a 2-glycidoxyethyl group, a 3-glycidoxypropyl group, or a 2-(3,4-epoxycyclohexyl)propyl group. As (meth)acryloxy group, included are a 3-methacryloxypropyl group, or a 3-acryloxypropyl group. As alkenyl group, included are a vinyl group, an allyl group, or an isopropenyl group. As mercaptoalkyl group, included are a mercaptopropyl group, or a mercaptoethyl group. As aminoalkyl group, included are a 3-(2-aminoethyl)aminopropyl group, a 3-aminopropyl group, or an N,N-dimethylaminopropyl group. As haloalkyl group, included are a 3-chloropropyl group, or a trifluoropropyl group. As glyceroxy group, included are a 3-glyceroxypropyl group, or a 2-glyceroxyethyl group. As ureido group, included are a 2-ureidoethyl group. As cyano group, included are a cyanopropyl group, or a cyanoethyl group.

Preferably, in the unit of formula (II), R¹ denotes a methyl group.

Advantageously, the organosilicon material contains units (I) and (II) in a molar ratio unit (I)/unit (II) in the range from 30/70 to 50/50, preferably in the range from 35/65 to 45/55.

The organosilicon particles can notably be obtained according to a method comprising:

(a) the introduction in an aqueous medium, in the presence of at least one hydrolysis catalyst, and optionally of at least one surfactant, of a compound (III) of formula SiX₄ and of a compound (IV) of formula RSiY₃, where X and Y denote, independently of one another, a C₁-C₄ alkoxy group, an alkoxyethoxy group containing a C₁-C₄ alkoxy group, a C₂-C₄ acyloxy group, an N,N-dialkylamino group containing a C₁-C₄ alkyl group, a hydroxyl group, a halogen atom or a hydrogen atom, and R denotes an organic group containing a carbon atom joined directly to the silicon atom; and

(b) bringing the mixture resulting from stage (a) into contact with an aqueous solution containing at least one polymerization catalyst and optionally at least one surfactant, at a temperature between 30 and 85° C., for at least two hours.

Stage (a) corresponds to a hydrolysis reaction and stage (b) corresponds to a reaction of condensation.

In stage (a), the molar ratio of compound (III) to compound (IV) is usually in the range from 30/70 to 50/50, advantageously from 35/65 to 45/55, and is preferably 40/60. The weight ratio of water to the sum of compounds (III) and (IV) is preferably in the range from 10/90 to 70/30. The order of introduction of compounds (III) and (IV) generally depends on their rate of hydrolysis. The temperature of the hydrolysis reaction is generally in the range from 0 to 40° C. and usually does not exceed 30° C. to avoid premature condensation of the compounds.

For groups X and Y of compounds (III) and (IV):

As C₁-C₄ alkoxy group, included are the methoxy and ethoxy groups;

As alkoxyethoxy group containing a C₁-C₄ alkoxy group, included are the methoxyethoxy and butoxyethoxy groups;

As C₂-C₄ alkoxy group, included are the acetoxy and propioxy groups;

As N,N-dialkylamino group containing a C₁-C₄ alkyl group, included are the dimethylamino and diethylamino groups;

As halogen atom, included are the atoms of chlorine and of bromine.

As compounds of formula (III), included are tetramethoxysilane, tetraethoxysilane, tetrabutoxysilane, trimethoxyethoxysilane, tributoxyethoxysilane, tetraacetoxysilane, tetrapropioxysilane, tetraacetoxysilane, tetra(dimethylamino)silane, tetra(diethylamino)silane, silane tetraol, chlorosilane triol, dichlorodisilanol, tetrachlorosilane, chlorotrihydrogenosilane. Preferably, the compound of formula (III) is selected from tetramethoxysilane, tetraethoxysilane, tetrabutoxysilane, and mixtures thereof.

The compound of formula (III) leads, after the polymerization reaction, to formation of the units of formula (I).

The compound of formula (IV) leads, after the polymerization reaction, to formation of the units of formula (II).

Group R in the compound of formula (IV) has the meaning as described for group R¹ for the compound of formula (II).

As examples of compounds of formula (IV) having an unreactive organic group R, included are methyltrimethoxysilane, ethyltriethoxysilane, propyltributoxysilane, butyltributoxysilane, phenyltrimethoxyethoxysilane, methyl tributoxyethoxysilane, methyltriacetoxysilane, methyltripropioxysilane, methyltriacetoxysilane, methyltri(dimethylamino)silane, methyltri(diethylamino)silane, methylsilane triol, methylchlorodisilanol, methyltrichlorosilane, methyltrihydrogenosilane.

As examples of compounds of formula (IV) having a reactive organic group R, included are:

the silanes having an epoxy group such as 3-glycidoxypropyl trimethoxysilane, 3-glycidoxypropyl triethoxysilane, 2-(3,4-epoxycyclohexyl)ethyl trimethoxysilane, 3-glycidoxypropylmethyl dimethoxysilane, 3-glycidoxypropylmethyl dimethoxysilane, 2-glycidoxyethylmethyldimethoxysilane, 3-glycidoxypropyl dimethylmethoxysilane, 2-glycidoxyethyl dimethylmethoxysilane;

the silanes having a (meth)acryloxy group such as 3-methacryloxypropyl trimethoxysilane, 3-acryloxypropyl trimethoxysilane;

the silanes having an alkenyl group such as vinyl trimethoxysilane, allyl trimethoxysilane, isopropenyl trimethoxysilane;

the silanes having a mercapto group such as mercaptopropyl trimethoxysilane, mercaptoethyl trimethoxysilane;

the silanes having an aminoalkyl group such as 3-aminopropyl trimethoxysilane, 3-(2-aminoethyl)aminopropyl trimethoxysilane, N,N-dimethylaminopropyl trimethoxysilane, N,N-dimethylaminoethyl trimethoxysilane;

the silanes having a haloalkyl group such as 3-chloropropyl trimethoxysilane, trifluoropropyl trimethoxysilane;

the silanes having a glyceroxy group such as 3-glyceroxypropyl trimethoxysilane, di(3-glyceroxypropyl)dimethoxysilane; the silanes having a ureido group such as 3-ureidopropyl trimethoxysilane, 3-ureidopropyl methyldimethoxysilane, 3-ureidopropyl dimethylmethoxysilane; the silanes having a cyano group such as cyanopropyl trimethoxysilane, cyanopropyl methyldimethoxysilane, cyanopropyl dimethylmethoxysilane.

Preferably, the compound of formula (IV) containing a reactive organic group R is selected from the silanes having an epoxy group, the silanes having a (meth)acryloxy group, the silanes having an alkenyl group, the silanes having a mercapto group, the silanes having an aminoalkyl group.

Examples of compounds (III) and (IV) that are preferred for application of this invention are tetraethoxysilane and methyltrimethoxysilane respectively.

Basic catalysts—such as sodium hydroxide, potassium hydroxide, sodium carbonate, sodium hydrogencarbonate, or amines (such as ammonia, trimethylamine, triethylamine, tetramethylammonium hydroxide)—or acid catalysts, selected from the organic acids—such as citric acid, acetic acid, methanesulphonic acid, p-toluene sulphonic acid, dodecylbenzenesulphonic acid, dodecylsulphonic acid—or mineral acids—such as hydrochloric acid, sulphuric acid, phosphoric acid, can be used independently as catalysts of hydrolysis and of polymerization. The surfactant, when present, is preferably a non-ionic or ionic surfactant or a mixture of the two. Sodium dodecylbenzenesulphonate can be used as anionic surfactant. The end of hydrolysis is indicated by the disappearance of products (III) and (IV), which are insoluble in water, and the formation of a homogeneous liquid layer.

Stage (b) of condensation can use the same catalyst as the hydrolysis stage or another catalyst selected from those mentioned above.

At the end of this procedure, we obtain an aqueous suspension of fine organosilicon particles, which can then optionally be separated from the medium. The method described above can therefore include an additional stage of filtration, for example on a membrane filter, of the product resulting from stage (b), optionally followed by a stage of centrifugation of the filtrate in order to separate the particles from the liquid medium, then a stage of drying of the particles. Other methods of separation can of course be used.

The form of the portions of hollow spheres obtained according to the method above, as well as their dimensions, will depend notably on the manner in which the products are brought into contact in stage (b).

A more basic pH and introduction of the polymerization catalyst in the mixture obtained from stage (a) in the cold will lead to portions of hollow spheres of a rounded-bottom “bowl” shape, whereas a more acid pH, and dropwise introduction of the mixture from stage (a) in the hot polymerization catalyst, will lead to portions of hollow spheres having a “horseshoe” shaped cross-section.

According to a preferred embodiment of the invention, portions of hollow spheres of “bowl” shape are used. These can be obtained as described in application JP-2003 128 788.

Portions of hollow spheres of horseshoe shape are also described in application JP-A-2000-191789.

The appended diagram shows a concave particle of portions of spheres of bowl shape in cross-section. The width W2 corresponds to the diameter of the particles. As can be seen from this diagram, these concave portions are formed (in section perpendicular to the plane of opening delimited by the portion of hollow sphere) from a small internal arc (11), a large external arc (21) and segments (31) which join together the ends of the respective arcs, the width (W1) between the two ends of the small internal arc (11) being in the range from 0.01 to 8 μm, preferably from 0.02 to 6 μm on average, the width (W2) between the two ends of the large external arc (21) being in the range from 0.05 to 10 μm, preferably from 0.06 to 8 μm on average and the height (H) of the large external arc (21) being in the range from 0.015 to 8 μm, preferably from 0.03 to 6 μm on average.

The dimensions stated above are obtained by calculating the average of the dimensions of a hundred particles selected from an image obtained with a scanning electron microscope.

As concave particles of portions of spheres that can be used according to the invention, included are:
particles comprising the crosslinked organosilicon TAK-110 (methylsilanol/silicate crosslinked polymer) of the company TAKEMOTO OIL & FAT, bowl-shaped, of width 2.5 μm, height 1.2 μm and thickness 150 nm (particles sold under the designation NLK-506 by the company Takemoto Oil & Fat)
particles comprising the crosslinked organosilicon TAK-110 (methylsilanol/silicate crosslinked polymer) of the company TAKEMOTO OIL & FAT, bowl-shaped, of width 0.8 μm, height 0.4 μm and thickness 130 nm (particles sold under the designation NLK-515 by the company Takemoto Oil & Fat)
particles comprising the crosslinked organosilicon TAK-110 (methylsilanol/silicate crosslinked polymer) of the company TAKEMOTO OIL & FAT, bowl-shaped, of width 7 μm, height 3.5 μm and thickness 200 nm (particles sold under the designation NLK-510 by the company Takemoto Oil & Fat)

These particles have the CTFA name: methylsilanol/silicate crosspolymer. Advantageously, the siliconized concave particles have an average diameter less than or equal to 5 μm, notably in the range from 0.1 μm to 5 μm, preferably in the range from 0.2 to 5 μm, more preferably in the range from 0.5 to 4 μm, and even more preferably in the range from 0.5 to 3 μm.

These particles of average diameter less than or equal to 5 μm make it possible to obtain thickening of the fat phase greater than that obtained with particles of average diameter greater than 5 μm. These particles provide optimization of the properties of slipperiness, spreading, and comfort of the composition according to the invention.

The siliconized concave particles can be present in the compositions of the kit according to the invention, preferably at a content in the range from 0.1 to 15 wt. %, relative to the total weight of the composition, preferably in the range from 0.5 to 10 wt. %, and more preferably in the range from 1 to 8 wt. %, including 0.5, 2, 4, 6, 8, 10, 12 and 14 wt. % and all values and subranges between stated values.

According to a preferred embodiment, the weight ratio of the content of siliconized concave particles to the content of silicone elastomer is in the range from 0.1 to 100, preferably from 0.2 to 50, and more preferably from 0.5 to 10.

Silicone Elastomers

The first composition of the kit according to the invention also contains a silicone elastomer.

According to a particular embodiment, the second composition of the kit according to the invention can also contain a silicone elastomer. It is to be appreciated, however, that the first and second compositions of the invention are not identical in all respects.

The silicone elastomer can be non-emulsifying or emulsifying.

According to a preferred embodiment, the first composition of the kit according to the invention can contain a non-emulsifying silicone elastomer.

The term “non-emulsifying” silicone elastomers defines organopolysiloxane elastomers not containing a hydrophilic chain such as polyoxyalkylene or polyglycerol units.

The non-emulsifying silicone elastomer is a crosslinked organopolysiloxane elastomer which can be obtained by a reaction of addition crosslinking of diorganopolysiloxane containing at least one hydrogen bound to the silicon and of diorganopolysiloxane having ethylenically-unsaturated groups bound to the silicon, notably in the presence of a platinum catalyst; or by a reaction of condensation crosslinking dehydrogenation between a hydroxyl-terminated diorganopolysiloxane and a diorganopolysiloxane containing at least one hydrogen bound to the silicon, notably in the presence of an organotin; or by a reaction of condensation crosslinking of a hydroxyl-terminated diorganopolysiloxane and a hydrolysable organopolysilane; or by thermal crosslinking of organopolysiloxane, notably in the presence of an organoperoxide catalyst; or by crosslinking of organopolysiloxane by high-energy radiation such as gamma rays, ultraviolet rays, electron beam.

Preferably, the crosslinked organopolysiloxane elastomer is obtained by a reaction of addition crosslinking (A2) of diorganopolysiloxane containing at least two hydrogens each bound to a silicon, and (B2) of diorganopolysiloxane having at least two ethylenically-unsaturated groups bound to the silicon, notably in the presence (C2) of a platinum catalyst, for example as described in application EP-A-295886.

In particular, the organopolysiloxane can be obtained by reaction of dimethylvinylsiloxy-terminated dimethylpolysiloxane and of trimethylsiloxy-terminated methylhydrogenopolysiloxane, in the presence of a platinum catalyst.

The compound (A2) is the base reactant for the formation of organopolysiloxane elastomer and crosslinking is carried out by a reaction of addition of compound (A2) with compound (B2) in the presence of the catalyst (C2).

Compound (A2) is advantageously a diorganopolysiloxane having at least two lower alkenyl groups (for example of C₂-C₄); the lower alkenyl group can be selected from the vinyl, allyl, and propenyl groups. These lower alkenyl groups can be situated at any position of the organopolysiloxane molecule but are preferably situated at the ends of the organopolysiloxane molecule. The organopolysiloxane (A2) can have a branched chain, linear chain, cyclic or network structure but the linear chain structure is preferred. Compound (A2) can have a viscosity in the range from the liquid state to the rubbery state. Preferably, compound (A2) has a viscosity of at least 100 centistokes at 25° C.

The organopolysiloxanes (A2) can be selected from methylvinylsiloxanes, methylvinylsiloxane-dimethylsiloxane copolymers, dimethylvinylsiloxy-terminated dimethylpolysiloxanes, dimethylsiloxane-dimethylvinylsiloxy-terminated methylphenylsiloxane copolymers, dimethylsiloxane-diphenylsiloxane-dimethylvinylsiloxy-terminated methylvinylsiloxane copolymers, dimethylsiloxane-trimethylsiloxy-terminated methylvinylsiloxane copolymers, dimethylsiloxane-methylphenylsiloxane-trimethylsiloxy-terminated methylvinylsiloxane copolymers, dimethylvinylsiloxy-terminated methyl(3,3,3-trifluoropropyl)polysiloxane, and dimethylsiloxane-dimethylvinylsiloxy-terminated methyl(3,3,3-trifluoropropyl)siloxane copolymers.

Compound (B2) is in particular an organopolysiloxane having at least 2 hydrogens bound to the silicon in each molecule and is therefore the crosslinking agent of compound (A2).

Advantageously, the sum of the number of ethylene groups per molecule of compound (A2) and the number of atoms of hydrogen bound to the silicon per molecule of compound (B2) is at least 4.

Compound (B2) can have any molecular structure, notably linear chain structure, branched chain structure, cyclic structure.

Compound (B2) can have a viscosity at 25° C. in the range from 1 to 50 000 centistokes, notably to have good miscibility with compound (A).

It is advantageous for compound (B2) to be added in an amount such that the molecular ratio between the total quantity of hydrogen atoms bound to the silicon in compound (B2) and the total quantity of all ethylenically-unsaturated groups in compound (A2) is in the range from 1/1 to 20/1.

Compound (B2) can be selected from trimethylsiloxy-terminated methylhydrogenopolysiloxanes, dimethylsiloxane/trimethylsiloxy-terminated methylhydrogenosiloxane copolymers, and dimethylsiloxane-methylhydrogenosiloxane cyclic copolymers.

Compound (C2) is the catalyst of the crosslinking reaction, and is notably chloroplatinic acid, chloroplatinic acid-olefin complexes, chloroplatinic acid-alkenylsiloxane complexes, chloroplatinic acid-diketone complexes, black platinum, and supported platinum.

Catalyst (C2) is preferably added from 0.1 to 1000 parts by weight, more preferably from 1 to 100 parts by weight, as platinum metal proper per 1000 parts by weight of the total quantity of compounds (A2) and (B2).

Other organic groups can be bound to the silicon in the organopolysiloxane (A2) and (B2) described previously, for example alkyl groups such as methyl, ethyl, propyl, butyl, octyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, an ester carboxylate group, a mercapto group.

The non-emulsifying silicone elastomer is generally mixed with at least one hydrocarbon oil and/or silicone oil to form a gel. In these gels, the non-emulsifying elastomer is in the form of non-spherical particles.

Non-emulsifying elastomers include those described in patents U.S. Pat. No. 4,970,252, U.S. Pat. No. 4,987,169, U.S. Pat. No. 5,412,004, U.S. Pat. No. 5,654,362, U.S. Pat. No. 5,760,116, and in application JP-A-61-194009.

As non-emulsifying elastomers, it is possible to use those sold under the designations “KSG-6”, “KSG-15”, “KSG-16”, “KSG-18”, “KSG-41”, “KSG-42”, “KSG-43”, “KSG-44”, “USG-105”, “USG-106” by the company Shin Etsu, “DC 9040”, “DC9041”, “DC 9509”, “DC 9505”, “DC 9506” by the company Dow Corning, “GRANSIL” by the company Grant Industries, “SFE 839” by the company General Electric.

The particles of crosslinked organopolysiloxane elastomers can also be in the form of powder, notably in the form of spherical powder.

The particles of silicone elastomer can also be in the form of powder of crosslinked organopolysiloxane elastomer coated with silicone resin, notably of silsesquioxane resin, as described for example in patent U.S. Pat. No. 5,538,793. Such elastomers are sold under the designations “KSP-100”, “KSP-101”, “KSP-102”, “KSP-103”, “KSP-104”, “KSP-105” by the company Shin Etsu.

Other silicone elastomers in the form of powders can be powders of hybrid silicone functionalized with fluoroalkyl groups, notably sold under the designation “KSP-200” by the company Shin Etsu; powders of hybrid silicones functionalized with phenyl groups, notably sold under the designation “KSP-300” by the company Shin Etsu.

The non-emulsifying silicone elastomer can be present in the compositions of the kit according to the invention preferably at a content in the range from 0.1 to 10 wt. %, relative to the total weight of the composition, preferably in the range from 0.5 to 7 wt. %, and more preferably in the range from 1 to 5 wt. %. including 1.5, 2, 4, 6, and 8 wt. % and all values and subranges between stated values.

The first and/or second composition can contain an emulsifying silicone elastomer.

By emulsifying silicone elastomer we mean a silicone elastomer containing at least one hydrophilic chain.

The emulsifying silicone elastomer can be selected from the polyoxyalkylenated silicone elastomers.

The polyoxyalkylenated silicone elastomer is a crosslinked organopolysiloxane which can be obtained by a reaction of addition crosslinking of diorganopolysiloxane containing at least one hydrogen bound to the silicon and of a polyoxyalkylene having at least two ethylenically-unsaturated groups.

Preferably, the crosslinked polyoxyalkylenated organopolysiloxane is obtained by a reaction of addition crosslinking (A1) of diorganopolysiloxane containing at least two hydrogens each bound to a silicon, and (B1) of polyoxyalkylene having at least two ethylenically-unsaturated groups, notably in the presence (C1) of a platinum catalyst, for example as described in patents U.S. Pat. No. 5,236,986 and U.S. Pat. No. 5,412,004.

In particular, the organopolysiloxane can be obtained by reaction of dimethylvinylsiloxy-terminated polyoxyalkylene (notably polyoxyethylene and/or polyoxypropylene) and trimethylsiloxy-terminated methylhydrogenopolysiloxane, in the presence of a platinum catalyst.

The organic groups bound to the silicon atoms of compound (A1) can be alkyl groups having from 1 to 18 carbon atoms, such as methyl, ethyl, propyl, butyl, octyl, decyl, dodecyl (or lauryl), myristyl, cetyl, stearyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, an ester carboxylate group, or a mercapto group.

Compound (A1) can thus be selected from trimethylsiloxy-terminated methylhydrogenopolysiloxanes, dimethylsiloxane/trimethylsiloxy-terminated methylhydrogenosiloxane copolymers, dimethylsiloxane-methylhydrogenosiloxane cyclic copolymers, dimethylsiloxane-methylhydrogenosiloxane-laurylmethylsiloxane trimethylsiloxy-terminated copolymers.

Compound (C1) is the catalyst of the crosslinking reaction, and is notably chloroplatinic acid, chloroplatinic acid-olefin complexes, chloroplatinic acid-alkenylsiloxane complexes, chloroplatinic acid-diketone complexes, black platinum, and supported platinum.

Advantageously, the polyoxyalkylenated silicone elastomers can be formed from divinylic compounds, in particular from polyoxyalkylenes having at least two vinyl groups, reacting with Si—H bonds of a polysiloxane.

The polyoxyalkylenated silicone elastomer according to the invention is carried in the form of a gel in at least one hydrocarbon oil and/or silicone oil. In these gels, the polyoxyalkylenated elastomer is in the form of non-spherical particles.

Polyoxyalkylenated elastomers are notably described in patents U.S. Pat. No. 5,236,986, U.S. Pat. No. 5,412,004, U.S. Pat. No. 5,837,793, U.S. Pat. No. 5,811,487 the contents of which are incorporated by reference.

As polyoxyalkylenated silicone elastomer, it is possible to use those marketed under the designations “KSG-21”, “KSG-20”, “KSG-30”, “KSG-31”, KSG-32”, “KSG-33”, “KSG-210”, “KSG-240”, “KSG-310”, “KSG-320”, “KSG-330”, “KSG-340”, “X-226146” by the company Shin Etsu, “DC9010”, “DC9011” by the company Dow Corning.

The emulsifying silicone elastomer can also be selected from the polyglycerol silicone elastomers.

The polyglycerol silicone elastomer is a crosslinked organopolysiloxane elastomer which can be obtained by a reaction of addition crosslinking of diorganopolysiloxane containing at least one hydrogen bound to the silicon and of polyglycerol compounds having ethylenically-unsaturated groups, notably in the presence of a platinum catalyst.

Preferably, the crosslinked organopolysiloxane elastomer is obtained by a reaction of addition crosslinking (A) of diorganopolysiloxane containing at least two hydrogens each bound to a silicon, and (B) of glycerol compounds having at least two ethylenically-unsaturated groups, notably in the presence (C) of a platinum catalyst.

In particular, the organopolysiloxane can be obtained by reaction of a dimethylvinylsiloxy-terminated polyglycerol compound and of trimethylsiloxy-terminated methylhydrogenopolysiloxane, in the presence of a platinum catalyst.

Compound (A) is the base reactant for the formation of organopolysiloxane elastomer and crosslinking is carried out by a reaction of addition of compound (A) with compound (B) in the presence of the catalyst (C).

Compound (A) is in particular an organopolysiloxane having at least 2 atoms of hydrogen bound to different silicon atoms in each molecule.

Compound (A) can have any molecular structure, notably a linear chain structure or branched chain structure or a cyclic structure.

Compound (A) can have a viscosity at 25° C. in the range from 1 to 50 000 centistokes, notably to have good miscibility with compound (B).

The organic groups bound to the silicon atoms of compound (A) can be alkyl groups having from 1 to 18 carbon atoms, such as methyl, ethyl, propyl, butyl, octyl, decyl, dodecyl (or lauryl), myristyl, cetyl, stearyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, an ester carboxylate group, or a mercapto group. Preferably, said organic group is selected from the methyl, phenyl and lauryl groups.

Compound (A) can thus be selected from the trimethylsiloxy-terminated methylhydrogenopolysiloxanes, the trimethylsiloxy-terminated dimethylsiloxane-methylhydrogenosiloxane copolymers, the dimethylsiloxane-methylhydrogenosiloxane cyclic copolymers, the trimethylsiloxy-terminated dimethylsiloxane-methylhydrogenosiloxane-laurylmethylsiloxane copolymers.

Compound (B) can be a polyglycerol compound corresponding to the following formula (B′):
CₘH₂ₘ₋₁—O—[Gly]n-CₘH₂ₘ₋₁ (B′)
in which m is an integer in the range from 2 to 6, n is an integer in the range from 2 to 200, preferably in the range from 2 to 100, preferably in the range from 2 to 50, preferably n is in the range from 2 to 20, preferably in the range from 2 to 10, and preferably in the range from 2 to 5, and in particular equal to 3; Gly denotes: —CH₂—CH(OH)—CH₂—O— or —CH₂—CH(CH₂OH)—O—

Advantageously, the sum of the number of ethylene groups per molecule of compound (B) and of the number of atoms of hydrogen bound to silicon atoms per molecule of compound (A) is of at least 4.

It is advantageous for compound (A) to be added in a quantity such that the molecular ratio between the total quantity of atoms of hydrogen bound to silicon atoms in compound (A) and the total quantity of all the ethylenically-unsaturated groups in compound (B) is in the range from 1/1 to 20/1.

Compound (C) is the catalyst of the crosslinking reaction, and is notably chloroplatinic acid, chloroplatinic acid-olefin complexes, chloroplatinic acid-alkenylsiloxane complexes, chloroplatinic acid-diketone complexes, black platinum, and supported platinum.

Catalyst (C) is preferably added from 0.1 to 1000 parts by weight, more preferably from 1 to 100 parts by weight, as platinum metal proper per 1000 parts by weight of the total quantity of compounds (A) and (B).

The polyglycerol silicone elastomer according to the invention is generally mixed with at least one hydrocarbon oil and/or silicone oil to form a gel. In these gels, the polyglycerol elastomer is often in the form of non-spherical particles.

Such elastomers are notably described in patent application WO2004/024798.

As polyglycerol silicone elastomers, it is possible to use those sold under the designations “KSG-710”, “KSG-810”, “KSG-820”, “KSG-830”, “KSG-840” by the company Shin Etsu.

The silicone elastomer (including the emulsifying and non-emulsifying elastomers) can be present in the first and/or second composition of the kit according to the invention at a content in the range from 0.1 to 10 wt. %, relative to the total weight of the composition, preferably in the range from 0.5 to 7 wt. %, and more preferably in the range from 1 to 5 wt. %.

The Oils

The compositions of the kit according to the invention can contain at least one oil.

The oil can be selected from volatile oils, non-volatile oils, and mixtures thereof.

The first and/or second composition according to the invention can contain at least one volatile oil.

By “volatile oil”, we mean in the sense of the invention any oil that can evaporate when in contact with the skin, at room temperature and atmospheric pressure. The volatile oils of the invention are cosmetic volatile oils, liquid at room temperature, having a non-zero vapour pressure, at room temperature and atmospheric pressure, in particular in the range from 0.13 Pa to 40 000 Pa (0.001 to 300 mmHg) and preferably in the range from 1.3 to 1300 Pa (0.01 to 10 mmHg).

The volatile oil can be selected from the hydrocarbon volatile oils, the silicone volatile oils, the fluorinated volatile oils, and mixtures thereof.

By “hydrocarbon oil”, we mean an oil containing mainly atoms of hydrogen and carbon and optionally atoms of oxygen, nitrogen, sulphur and/or phosphorus.

The volatile hydrocarbon oils can be selected from the hydrocarbon oils having from 8 to 16 carbon atoms, and notably the C₈-C₁₆ branched alkanes such as the C₈-C₁₆ isoalkanes of petroleum origin (also called isoparaffins) such as isododecane (also called 2,2,4,4,6-pentamethylheptane), isodecane, isohexadecane, and for example the oils sold under the trade names Isopars® or Permethyls®.

Volatile silicones can also be used as volatile oils, for example oils of linear or cyclic volatile silicones, notably those having a viscosity≦5 centistokes (5×10⁻⁶ m²/s), and having notably from 2 to 10 silicon atoms, preferably from 2 to 7 silicon atoms, these silicones optionally containing alkyl or alkoxy groups having from 1 to 10 carbon atoms. As oil of volatile silicone that can be used in the invention, included are notably octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, 3-butyl 1,1,1,3,5,5,5-heptamethyl trisiloxane, 3-propyl 1,1,1,3,5,5,5-heptamethyl trisiloxane, 3-ethyl 1,1,1,3,5,5,5-heptamethyl trisiloxane, decamethyl tetrasiloxane, dodecamethyl pentasiloxane and mixtures thereof.

The fluorinated volatile oil does not generally have a flash point.

As fluorinated volatile oil, included are nonafluoroethoxybutane, nonafluoromethoxybutane, decafluoropentane, tetradecafluorohexane, dodecafluoropentane, and mixtures thereof.

The volatile oils are present in the first and/or second composition of the kit according to the invention at a content in the range from 6 to 45 wt. % relative to the total weight of the composition, preferably in the range from 6 to 40 wt. % and more preferably in the range from 6 to 35 wt. %.

The first and/or second composition according to the invention can contain at least one non-volatile oil.

By “non-volatile oil”, we mean an oil that remains on the skin at room temperature and atmospheric pressure for at least several hours and notably having a vapour pressure less than 0.13 Pa (0.01 mmHg).

These non-volatile oils can be hydrocarbon oils notably of animal or vegetable origin, silicone oils, or mixtures thereof. By “hydrocarbon oil”, we mean an oil containing mainly atoms of hydrogen and carbon and optionally atoms of oxygen, of nitrogen, of sulphur and/or of phosphorus.

The non-volatile oils can notably be selected from hydrocarbon oils, possibly fluorinated, and/or non-volatile silicone oils.

We may notably mention, as non-volatile hydrocarbon oil:
hydrocarbon oils of animal origin,
hydrocarbon oils of vegetable origin such as triglycerides comprising esters of fatty acids and of glycerol where the fatty acids can have chain lengths varying from C₄ to C₂₄, and the latter can be linear or branched, saturated or unsaturated; these oils are notably triglycerides of heptanoic acid or of octanoic acid, or alternatively wheat germ oil, sunflower oil, grapeseed oil, maize oil, apricot oil, castor oil, karite oil, avocado oil, olive oil, soya oil, sweet almond oil, palm oil, cottonseed oil, hazelnut oil, macadamia nut oil, jojoba oil, lucerne oil, poppy oil, Chinese okra oil, sesame oil, cucurbit oil, colza oil, black currant oil, evening primrose oil, millet oil, barley oil, quinoa oil, rye oil, safflower oil, kukui nut oil, passionflower oil, musk rose oil; shea butter; or triglycerides of caprylic/capric acids such as those sold by the company Stéarineries Dubois or those sold under the designations Miglyol 810®, 812® and 818® by the company Dynamit Nobel,
synthetic ethers having from 10 to 40 carbon atoms;
linear or branched hydrocarbons, of mineral origin or synthetic, such as Vaseline, polydecenes, hydrogenated polyisobutene such as Parleam®, squalane, paraffin oils, and mixtures thereof,
synthetic esters such as the oils of formula R₁COOR₂ in which R₁ represents the residue of a linear or branched fatty acid containing from 1 to 40 carbon atoms and R₂ represents a hydrocarbon chain notably branched containing from 1 to 40 carbon atoms with the proviso that R₁+R₂≧10, for example Purcellin oil (ketostearyl octanoate), isopropyl myristate, isopropyl palmitate, the benzoates of C₁₂ to C₁₅ alcohols, hexyl laurate, diisopropyl adipate, isononyl isononanoate, isodecyl neopentanoate, 2-ethyl-hexyl palmitate, isostearyl isostearate, 2-hexyl-decyl laurate, 2-octyl-decyl palmitate, 2-octyl-dodecyl myristate, the heptanoates, octanoates, decanoates or ricinoleates of alcohols or of polyalcohols such as propyleneglycol dioctanoate; the hydroxylated esters such as isostearyl lactate, di-isostearyl malate, 2-octyl-dodecyl lactate; the esters of polyols and the esters of pentaerythritol,
the aliphatic alcohols that are liquid at room temperature, with a branched carbon chain and/or unsaturated having from 12 to 26 carbon atoms such as octyl dodecanol, isostearyl alcohol, oleic alcohol, 2-hexyldecanol, 2-butyloctanol, and 2-undecylpentadecanol,
the higher fatty acids such as oleic acid, linoleic acid, linolenic acid and mixtures thereof.

The non-volatile silicone oils that can be used in the composition according to the invention include the non-volatile polydimethylsiloxanes (PDMS), the polydimethylsiloxanes with pendent alkyl or alkoxy groups and/or at the ends of the silicone chain, groups each with from 2 to 24 carbon atoms, the phenylated silicones such as the phenyl trimethicones, the phenyl dimethicones, the phenyl trimethylsiloxy diphenylsiloxanes, the diphenyl dimethicones, the diphenyl methyldiphenyl trisiloxanes, and mixtures thereof. Preferred non-volatile hydrocarbon oils used include the esters of formula R₁COOR₂ in which R₁, represents the residue of a branched fatty acid having from 6 to 10 carbon atoms and R₂ represents a hydrocarbon chain (or a mixture of hydrocarbon chains) containing from 10 to 18 carbon atoms, preferably from 12 to 16 carbon atoms.

The oil (or the mixture of oils) of the first and/or second composition according to the invention can be present at a content for example in the range from 1 to 80 wt. %, relative to the total weight of the composition, preferably in the range from 5 to 60 wt. %, and more preferably from 5 to 50 wt. %.

According to a preferred embodiment, the first composition of the kit according to the invention contains at least one volatile oil.

According to a particular embodiment, the second composition according to the invention contains at least one volatile silicone oil and a non-volatile oil of a C₁₀-C₁₈ alkyl ester, in particular a C₁₂-C₁₅ alkyl ester.

According to a preferred embodiment, the C₁₀-C₁₈ alkyl ester (in particular C₁₂-C₁₅) can be present in the second composition at a content in the range from 5 to 20 wt. %, relative to the total weight of the composition.

Aqueous Phase

The compositions of the kit according to the invention can contain an aqueous phase.

The aqueous phase contains water. The water can be any water, such as a floral water such as cornflower water and/or a mineral water such as Vittel water, Lucas water or La Roche Posay water and/or a thermal water.

The aqueous phase can contain at least one water-miscible organic solvent (at room temperature −25° C.) for example monohydric alcohols having from 2 to 6 carbon atoms such as ethanol, isopropanol; polyols notably having from 2 to 20 carbon atoms, preferably having from 2 to 10 carbon atoms, and more preferably having from 2 to 6 carbon atoms, such as glycerol, propylene glycol, butylene glycol, pentylene glycol, hexylene glycol, dipropylene glycol, diethylene glycol; glycol ethers (notably having from 3 to 16 carbon atoms) such as alkyl(C₁-C₄)ether of mono-, di- or tripropylene glycol, alkyl(C₁-C₄)ethers of mono-, di- or triethylene glycol, and mixtures thereof.

According to a preferred embodiment, the aqueous phase of the first and/or of the second composition can contain a hydrating polyol selected from the polyols preferably having from 2 to 10 carbon atoms, and more preferably having from 2 to 6 carbon atoms. In particular, the water-miscible organic solvent is selected from glycerol, propylene glycol, butylene glycol, and mixtures thereof.

The first and/or the second composition of the kit according to the invention can contain at least one hydrating polyol at a content less than or equal to 15 wt. %, relative to the total weight of each composition, preferably in the range from 4 to 15 wt. %, more preferably from 5 to 12 wt. %, and even more preferably in the range from 8 to 12 wt. %.

According to a more preferred embodiment, each of the first and second composition of the kit according to the invention contains at least one hydrating polyol.

According to a particular embodiment, the sum of the contents of hydrating polyols of the first and second compositions, expressed by weight, relative to the total weight of each composition, is greater than or equal to 8, and is in particular in the range from 8 to 30, preferably from 10 to 25, and more preferably from 15 to 20.

The aqueous phase can additionally contain stabilizers, for example sodium chloride, magnesium dichloride and magnesium sulphate.

The aqueous phase can also contain any water-soluble or water-dispersible compound compatible with an aqueous phase, such as gelling agents, film-forming polymers, thickeners, surfactants and mixtures thereof.

Preferably, the aqueous phase can be present in the first and/or the second composition of the kit according to the invention at a content in the range from 5 to 80 wt. % relative to the total weight of the composition and preferably in the range from 10 to 70 wt. %.

Preferably, water can be present in the first and/or the second composition according to the invention at a content in the range from 10 to 75 wt. % relative to the total weight of the composition, preferably in the range from 15 to 65 wt. %.

Colourants

The compositions of the kit according to the invention, preferably at least the second composition, can contain a pulverulent colourant. According to a particular embodiment, the first composition can contain a pulverulent colourant.

By colourant, in the sense of the present invention, we mean a compound that can produce a coloured optical effect when it is formulated in sufficient quantity in a suitable medium.

The pulverulent colourant can notably be selected from pigments, nacres, and mixtures thereof.

“Pigments” is to be understood as meaning white or coloured, mineral or organic particles, insoluble in the liquid organic phase, intended to colour and/or opacify the composition. “Nacres” is to be understood as meaning iridescent particles, notably produced in the shells of certain mollusks or synthesized, which are insoluble in the composition.

According to one embodiment of the invention, the colourant contains at least one pigment.

The pigments can be selected from mineral pigments, organic pigments, and composite pigments (i.e. pigments based on mineral and/or organic materials).

“Pigments” is to be understood as meaning particles of any form, endowed with an optical effect, mineral or synthetic, insoluble in the medium of the composition regardless of the temperature at which the composition is manufactured.

The pigments can be selected from monochrome pigments, lakes, nacres, pigments with optical effects, such as reflective pigments and goniochromatic pigments.

The mineral pigments can be selected from metal oxide pigments, mica coated with titanium dioxide, mica coated with bismuth oxychloride, titanium mica coated with iron oxide, titanium mica coated with ferric blue, titanium mica coated with chromium oxide, iron oxides, titanium dioxide, zinc oxides, cerium oxide, zirconium oxide, chromium oxide; manganese violet, Prussian blue, ultramarine, ferric blue, bismuth oxychloride, coloured lustre pigments such as titanium mica with iron oxides, titanium mica notably with ferric blue or chromium oxide, titanium mica with an organic pigment of an aforementioned type as well as lustre pigments based on bismuth oxychloride, and mixtures thereof.

The organic pigments can be for example:
cochineal carmine,
organic pigments of azo, anthraquinone, indigoid, xanthene, pyrene, quinoline dyes, of triphenylmethane, of fluorane;
organic lakes or insoluble sodium, potassium, calcium, barium, aluminium, zirconium, strontium or titanium salts, acid dyes such as the azo, anthraquinone, indigoid, xanthene, pyrene, quinoline dyes, triphenylmethane, fluorane. These colourants generally have at least one carboxylic or sulphonic acid group;
melanic pigments.

Among the organic pigments, included are D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

According to a preferred embodiment, the pigments present in the first and/or second compositions according to the invention are hydrophobic coated pigments. “Hydrophobic coated pigments” means pigments surface-treated with a hydrophobic agent to make them compatible with the oil phase of the emulsion, notably so that they have good wettability with the oils of the oil phase. Thus, these treated pigments are well dispersed in the oil phase.

The pigments that are to be coated can be mineral or organic pigments as described above.

Pigments of iron oxides or of titanium dioxide are preferably used.

The agent for hydrophobic treatment can be selected from silicones such as methicones, dimethicones, perfluoroalkylsilanes; fatty acids such as stearic acid; metallic soaps such as aluminium dimyristate, the aluminium salt of glutamate of hydrogenated tallow, perfluoroalkyl phosphates, perfluoroalkyl silanes, perfluoroalkyl silazanes, of hexafluoropropylene polyoxides, polyorganosiloxanes containing perfluoroalkyl perfluoropolyether groups, amino acids; N-acylated amino acids or their salts; lecithin, isopropyl trisostearyl titanate, and mixtures thereof.

The N-acylated amino acids can contain an acyl group having from 8 to 22 carbon atoms, for example a 2-ethyl hexanoyl, caproyl, lauroyl, myristoyl, palmitoyl, stearoyl, or cocoyl group. The salts of these compounds can be aluminium, magnesium, calcium, zirconium, zinc, sodium, or potassium salts. The amino acid can be for example lysine, glutamic acid, alanine.

The term alkyl used in the aforementioned compounds denotes notably an alkyl group having from 1 to 30 carbon atoms, preferably having from 5 to 16 carbon atoms.

Hydrophobic treated pigments are notably described in application EP-A-1086683. The first and/or second composition of the kit according to the invention can include nacres. The nacres can be selected from the white lustre pigments such as mica coated with titanium dioxide or bismuth oxychloride, coloured lustre pigments such as titanium mica coated with iron oxides, titanium mica coated notably with ferric blue or chromium oxide, titanium mica coated with an organic pigment of the type mentioned above as well as the lustre pigments based on bismuth oxychloride.

The pigments and/or the nacres can preferably be present in the composition(s), preferably the first composition, at a content in the range from 0.01 to 6 wt. %, relative to the total weight of the first composition, preferably in the range from 0.5 to 5 wt. %, and more preferably in the range from 1 to 3 wt. %.

According to a preferred embodiment, the pulverulent colourant of the first composition of the kit according to the invention comprises at least one nacre.

According to a preferred embodiment, the first composition of the kit according to the present invention contains less than 5 wt. %, relative to the total weight of the composition, of pigments, notably from 0.1 to 5 wt. %, preferably from 0.1 to 3 wt. % of pigments.

According to a preferred embodiment, the first composition of the kit according to the present invention does not contain a pigment.

The pigments and/or the nacres can be present in the second composition at a content in the range from 0.01 to 20 wt. %, relative to the total weight of the first composition, and preferably in the range from 0.5 to 18 wt. %, and more preferably in the range from 3 to 15 wt. %.

The colouring matter can also include water-soluble or oil-soluble colourants. The pulverulent colouring matter can be present in the first and/or second composition at a content in the range from 0.01 to 25 wt. %, relative to the total weight of the composition, preferably in the range from 0.5 to 20 wt. %, and more preferably in the range from 1 to 18 wt. %.

Fillers

The compositions of the kit according to the invention can contain fillers.

“Fillers” is intended to mean particles of any shape, colored, colourless or white, mineral or synthetic, insoluble in the medium of the composition regardless of the temperature at which the composition is manufactured.

The fillers can be mineral or organic, of any shape, lamellar, spherical or oblong, whatever the crystallographic form (for example flake, cubic, hexagonal, orthorhombic, etc.). Included are talc, mica, silica, kaolin, polyamide (Nylon®) powders, poly-β-alanine powders, polyethylene powders, polyurethane powders such as powder of copolymer of hexamethylene diisocyanate and trimethylol hexyl lactone sold under the designation PLASTIC POWDER D400 by the company TOSHIKI, powders of tetrafluoroethylene polymers (Teflon®), lauroyl-lysine, starch, boron nitride, hollow polymer microspheres such as those of polyvinylidene chloride/acrylonitrile such as Expancel® (Nobel Industrie), acrylic acid copolymers, particles of polymethyl methacrylate, precipitated calcium carbonate, magnesium carbonate and hydrocarbonate, hydroxyapatite, hollow silica microspheres, glass or ceramic microcapsules, metallic soaps derived from organic carboxylic acids having from 8 to 22 carbon atoms, preferably from 12 to 18 carbon atoms, for example zinc, magnesium or lithium stearate, zinc laurate, magnesium myristate; barium sulphate and mixtures thereof.

The fillers can be present, for example, at a content in the range from 0.5 to 25 wt. %, relative to the total weight of each composition in which it (they) are present, preferably in the range from 1 to 20 wt. %, and more preferably in the range from 2 to 15 wt. %.

According to a preferred embodiment, the second composition of the kit according to the invention contains less than 5 wt. %, relative to the total weight of the composition, of absorbent fillers, notably from 0.1 to 5 wt. %, and preferably from 0.1 to 3 wt. %. According to a more preferred embodiment, the second composition of the kit according to the invention does not contain any absorbent fillers.

Absorbent fillers or “sebum pumps” in the sense of the present application means a compound that is able to absorb and/or adsorb sebum.

Generally this type of compound is in the form of a powder having a sebum uptake. Advantageously, the sebum uptake of these compounds is greater than or equal to 1 ml/g, and can notably vary from 1 ml/g to 20 ml/g, in particular from 1 ml/g to 15 ml/g. It can notably be greater than or equal to 1.5 ml/g, and in particular can vary from 1.5 ml/g to 20 ml/g, or vary from 1.5 ml/g to 15 ml/g.

Such fillers are notably described in patent application EP-A-1559393 the content of which is incorporated by reference in the present application.

They can notably be selected from: silica, polyamide (nylon) powders, powders of acrylic polymers, notably of polymethyl methacrylate, of polymethyl methacrylate/ethylene glycol dimethacrylate, of allyl polymethacrylate/ethylene glycol dimethacrylate 5 acrylate, ethylene glycol dimethacrylate/lauryl methacrylate copolymer and polyethylene powders, notably polyethylene/acrylic acid powders.

As representative and non-limiting examples of absorbent fillers according to the invention, included are quite particularly:
silica powders, for example the porous silica microspheres sold under the designation “SILICA BEADS SB-700” marketed by the company MYOSHI, the “SUNSPHERE H51”, “SUNSPHERE H33”, “SUNSPHERE H53” marketed by the company ASAHI GLASS; and the coated amorphous silica microspheres of “polydimethylsiloxane 5 ne” sold under the designation “SA SUNSPHERE H-33” and “SA SUNSPHERE H-53” by the company ASAHI GLASS,
polyamide (nylon) powders, for example “ORGASOL 4000” or “ORGASOL 2002 EXTRA D NAT COS” marketed by the company ATOCHEM,
powders of acrylic polymers, notably of polymethyl methacrylate, for example “COVABEAD□ LH85” marketed by the company WACKHERR; of polymethyl methacrylate/ethylene glycol dimethacrylate, for example “DOW CORNING 5640 MICROSPONGE□ SKIN OIL ADSORBER” marketed by the company DOW CORNING, or “GANZPEARL□ GMP-0820” marketed by the company GANZ CHEMICAL; of allyl polymethacrylate/ethylene glycol dimethacrylate, for example “POLY-PORE□ L200” or “POLY-PORE E200” marketed by the company AMCOL; of ethylene glycol dimethacrylate/lauryl methacrylate copolymer, for example “POLYTRAP 6603” marketed by the company DOW CORNING, and
powders of polyethylene, notably of polyethylene/acrylic acid sold under the trade name Flobeads® by the company SUMITOMO.
   Viscosity of the Second Composition of the Kit

Advantageously, the second composition according to the invention has a viscosity, measured at 25° C., at a shear gradient of 200 s⁻¹, in the range from 0.1 to 0.8 Pa·s (1 to 8 poise), and preferably in the range from 0.2 to 0.7 Pa·s (2 to 7 poise), including 0.3, 0.4, 0.5 and 0.6 Pa·s including all values and subranges between stated values. Such a viscosity gives good sliding properties on application of the composition on the skin. The viscosity is measured with a RHEOSTRESS RS 600 controlled-stress rheometer of the company THERMO using a sandblasted plane cone of 60 mm, having an angle of 2° with a gap of 0.105 mm.

Galenical

The first composition of the kit according to the invention is preferably in the form of a water-in-oil or oil-in-water emulsion, or of an anhydrous composition (powder, stick, oily gel) and preferably water-in-oil.

The second composition of the kit is preferably in the form of an emulsion (oil-in-water or water-in-oil), of an aqueous gel or anhydrous and preferably water-in-oil.

According to a particular embodiment, the continuous phases of the first and second compositions are of the same nature.

According to a more particular embodiment, the continuous phases of the first and second compositions are oily.

According to a preferred embodiment, the second composition is in the form of a water-in-oil emulsion.

According to a preferred embodiment, the kit according to the invention can contain:

i) A first composition in the form of water-in-oil emulsion comprising:

0.1 to 10 wt. %, relative to the total weight of the composition, of at least one silicone elastomer,
0.1 to 15 wt. %, relative to the total weight of the composition, of concave particles of a siliconized material, notably in the form of portions of hollow spheres
at least one hydrating polyol, and
containing less than 5 wt. %, relative to the total weight of the composition, of pigments; and
   ii) A second composition in the form of water-in-oil emulsion comprising:
0.01 to 20 wt. %, relative to the total weight of the composition, of pigments and/or nacres,
at least one hydrating polyol, and
containing less than 5 wt. %, relative to the total weight of the composition, of absorbent fillers.
   Additives

The compositions according to the invention can contain at least one other cosmetic ingredient that can be selected notably from gelling agents and/or hydrophilic or lipophilic thickeners, antioxidants, perfumes, preservatives, neutralizing agents, sun filters, vitamins, self-tanning compounds, antiwrinkle actives, emollients, waxes, hydrophilic or lipophilic actives, anti-pollution agents or anti-free radical agents, sequestering agents, film-forming agents, surfactants, dermorelaxant actives, calmatives, agents stimulating the synthesis of dermal or epidermal macromolecules and/or preventing their degradation, anti-glycation agents, anti-irritant agents, desquamating agents, depigmenting, anti-pigmenting, or pro-pigmenting agents, inhibitors of NO-synthase, agents stimulating the proliferation of fibroblasts or of keratinocytes and/or differentiation of keratinocytes, agents acting on the microcirculation, agents acting on the energy metabolism of cells, healing agents, and mixtures thereof.

The invention is described in more detail in the examples given below. Contents are expressed as percentages by weight.

### EXAMPLES OF MAKE-UP KITS

### Examples 1 and 2

### First Compositions Of The Kit According To The Invention

| | |
|---|---|
| Ex. 1 | Ex. 2 |

Oil phase

| | | |
|---|---|---|
| Gel of silicone elastomer - dimethicone/ | 10 | 12.6 |
| vinyl dimethicone copolymer (25%) in | | |
| polydimethylsiloxane 6 cSt sold under | | |
| the designation KSG-16 by the company | | |
| SHIN ETSU | | |
| Cyclopentasiloxane | 18.25 | 18.05 |
| Polydimethylsiloxane 10 cSt | 0 | 2 |
| Isododecane | 4.6 | 0 |
| Mixture of cetyl dimethicone copolyol, | 9 | 9 |
| polyglyceryl-4 isostearate and hexyl | | |
| laurate (30/40/30) sold under the | | |
| designation Abil WE 09 by the company | | |
| GOLDSCHMIDT | | |
| Siliconized concave particles sold under | 4 | 4 |
| the designation NLK-506 by the company | | |
| TAKEMOTO OILS & FAT | | |
| Hollow microspheres of polymethyl | 5 | 5 |
| methacrylate sold under the name | | |
| COVABEAD LH85 by the company LCW | | |
| Nacre | 0 | 0.2 |

Aqueous phase

| | | |
|---|---|---|
| Preservative | qs | qs |
| Propylene glycol | 5 | 5 |
| Acetylated ethylene glycol stearate | 0.7 | 0.7 |
| Magnesium sulphate | 0.7 | 0.7 |
| Water | qsf 100% | qsf 100% |

Procedure:

Melt the silicone elastomer gel, the mixture of cetyl dimethicone copolyol, polyglyceryl-4 isostearate and hexyl laurate and the acetylated ethylene glycol stearate. Add the cyclopentasiloxane, isododecane and polydimethylsiloxane. Then disperse the PMMA microspheres, the siliconized concave particles and the nacre in the mixture. The mixture obtained then constitutes the oil phase of the emulsion. Mix the other constituents separately (aqueous phase), then pour this mixture into the oil phase according to a classical method of emulsification in a Moritz homogenizer.

### Examples 3 to 6

### Second Compositions of the Kit According to the Invention

| | | | |
|---|---|---|---|
| Exam- | Exam- | Exam- | Exam- |
| ple 3 | ple 4 | ple 5 | ple 6 |

Oil phase

| | | | | |
|---|---|---|---|---|
| Cetyl dimethicone copolyol | 0.80 | 0.80 | 0.80 | 0.80 |
| sold under the designation | | | | |
| Abil EM 90 by the company | | | | |
| GOLDSCHMIDT | | | | |
| Polyglyceryl-4 isostearate | 0.60 | 0.60 | 0.60 | 0.6 |
| sold under the designation | | | | |
| Isolan GI 34 by the | | | | |
| company GOLDSCHMIDT | | | | |
| Hexyl laurate | 0.60 | | | |
| Oxyethylenated polydimeth- | 5.0 | 5.0 | 5.0 | 5.0 |
| ylsiloxane sold under the | | | | |
| designation KF-6017 by the | | | | |
| company Shin Etsu | | | | |
| Isoeicosane | 4.50 | | | |
| Dimethicone (5 cSt) | 2.40 | | | |
| Isostearyl neopentanoate | 0.50 | | | |
| Isononyl isononanoate | | | 19.20 | 10.00 |
| Dicaprylic ether | | 19.20 | | |
| Cyclohexasiloxane | 5.90 | | | |
| Cyclopentane | 10.40 | 18.0 | 18.0 | 18.0 |
| Ethyl 2-hexanoate of C₁₂- | | | | 9.20 |
| C₁₅ alkyl sold under the | | | | |
| designation Hetester by | | | | |
| the company Heterene | | | | |
| Chemical | | | | |
| Modified hectorite sold | 1.90 | 1.90 | 1.90 | 1.90 |
| under the designation | | | | |
| Bentone 38 VCG by the | | | | |
| company Elementis | | | | |
| Isododecane | 13.40 | | | |
| Vitamin E | 0.10 | 0.10 | 0.10 | 0.10 |
| Coated pigments | 11.0 | 11.0 | 11.0 | 11.00 |
| Microbeads of polymethyl- | 4.0 | | | |
| silsesquioxane resin sold | | | | |
| under the designation | | | | |
| TOSPEARL 145 A by the | | | | |
| company GE Toshiba | | | | |
| Silicones | | | | |
| Hollow microspheres of | | 2.0 | 2.0 | 2.0 |
| polymethyl methacrylate | | | | |
| sold under the name | | | | |
| COVABEAD LH85 by the | | | | |
| company LCW | | | | |

Aqueous phase

| | | | | |
|---|---|---|---|---|
| Butylene glycol | 3.0 | 3.0 | 3.0 | 3.0 |
| Glycerol | 7.0 | 7.0 | 7.0 | 7.0 |
| Sodium chloride | 0.70 | 0.70 | 0.70 | 0.70 |
| D-Panthenol | 0.10 | 0.10 | 0.10 | 0.10 |
| Preservative | qs | qs | qs | qs |
| Water | qsf | qsf | qsf | qsf |
| | 100% | 100% | 100% | 100% |

Procedure:
Preparation of a Pigment Paste:

Pulverized pigments are prepared in a portion of cyclopentasiloxane.

Preparation of the Oil Phase:

Mix phases A1 and A2 at room temperature in a Moritz homogenizer.

Add the hectorite to the mixture A1+A2 and continue stirring until a homogeneous mixture is obtained. Then add the pulverized pigments and the vitamin E and the fillers, stirring in the Moritz for 10 min until the microbeads are dispersed homogeneously.

Preparation of the Aqueous Phase:

Mix the constituents of B1 at room temperature.

Preparation of the Emulsion:

While stirring in the Moritz at room temperature, pour the aqueous phase into the oil phase. Continue stirring until the aqueous phase is well dispersed in the oil phase.

Apply one or the other of the compositions in Examples 1 or 2 to the skin. Application of this first composition on the skin gives good camouflaging of skin blemishes.

Then apply one of the compositions in Examples 3, 4 or 5 (second composition of the kit) on the first deposit.

The make-up obtained is uniform, it masks the skin blemishes and avoids accentuating the wrinkles or lines.

The above written description of the invention provides a manner and process of making and using it such that any person skilled in this art is enabled to make and use the same, this enablement being provided in particular for the subject matter of the appended claims, which make up a part of the original description and including a skin make-up kit, comprising a first and a second cosmetic composition, the first composition comprising at least one silicone elastomer and concave particles of a siliconized material, notably in the form of portions of hollow spheres, the second composition comprising a physiologically acceptable medium.

As used herein, the phrases “selected from the group consisting of,” “chosen from,” and the like include mixtures of the specified materials. Terms such as “contain(s)” and the like as used herein are open terms meaning “including at least” unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

In this regard, the invention method and composition is preferably used by subjects desirous of the benefits noted herein, subjects “in need of” these benefits. Such subjects are typically desirous to obtain make-up that is uniform, and/or masks skin blemishes and/or avoids accentuating wrinkles or lines.

1. A kit comprising a first and a second composition, the first composition comprising at least one silicone elastomer and siliconized concave particles, the second composition being different from the first and comprising a physiologically acceptable medium.

2. The kit according to claim 1, wherein said siliconized concave particles are in the form of portions of hollow spheres and have an average diameter less than or equal to 10 μm.

3. The kit according to claim 1, wherein said siliconized concave particles have an average diameter of 0.05 to 10 μm.

4. The kit according to claim 1, wherein the siliconized concave particles are in the form of portions of hollow spheres having a cross-section in the shape of a horseshoe or an arch.

5. The kit according to claim 1, wherein the siliconized concave particles comprise a crosslinked polysiloxane with a three-dimensional structure comprising units of formula (I): SiO₂
and of formula (II): R¹SiO_{1.5}
in which R¹ denotes an organic group having a carbon atom joined directly to the silicon atom.

6. The kit according to claim 5, wherein the organic group is an unreactive organic group.

7. The kit according to claim 6, wherein the unreactive organic group is a C₁-C₄ alkyl group or a phenyl group.

8. The kit according to claim 7, wherein the unreactive organic group is a methyl group.

9. The kit according to claim 1, wherein the organic group is a reactive group selected from an epoxy group, a (meth)acryloxy group, an alkenyl group, a mercaptoalkyl group, an aminoalkyl group, a haloalkyl group, a glyceroxy group, a ureido group, and a cyano group.

10. The kit according to claim 9, wherein the reactive organic group is selected from an epoxy group, a (meth)acryloxy group, an alkenyl group, a mercaptoalkyl group, or an aminoalkyl group.

11. The kit according to claim 5, wherein R¹ denotes a methyl group.

12. The kit according to claim 5, wherein the siliconized concave particles comprise a crosslinked polysiloxane having units (I) and (II) in a molar ratio unit (I)/unit (II) in the range from 30/70 to 50/50.

13. The kit according to claim 1, wherein the siliconized concave particles are obtained according to a method comprising:
(a) the introduction in an aqueous medium, in the presence of at least one hydrolysis catalyst, and optionally of at least one surfactant, of a compound (III) of formula SiX₄ and of a compound (IV) of formula RSiY₃ to form a mixture, where X and Y denote, independently of one another, a C₁-C₄ alkoxy group, an alkoxyethoxy group containing a C₁-C₄ alkoxy group, a C₂-C₄ acyloxy group, an N,N-dialkylamino group containing a C₁-C₄ alkyl group, a hydroxyl group, a halogen atom or a hydrogen atom, and R denotes an organic group with a carbon atom joined directly to the silicon atom; and
(b) bringing the mixture resulting from stage (a) into contact with an aqueous solution containing at least one polymerization catalyst and optionally at least one surfactant, at a temperature between 30 and 85° C., for at least two hours.

14. The kit according to claim 13, wherein in stage (a) the molar ratio of compound (III) to compound (IV) is in the range from 30/70 to 50/50.

15. The kit according to claim 13, wherein the weight ratio of water to the sum of compounds (III) and (IV) is in the range from 10/90 to 70/30 in stage (a).

16. The kit according to claim 13, wherein R is a reactive organic group.

17. The kit according to claim 13, wherein R is an unreactive organic group selected from a C₁-C₄ alkyl group and a phenyl group.

18. The kit according to claim 17, wherein the unreactive organic group is a methyl group.

19. The kit according to claim 16, wherein the reactive organic group is selected from an epoxy group, a (meth)acryloxy group, an alkenyl group, a mercaptoalkyl group, an aminoalkyl group, a haloalkyl group, a glyceroxy group, a ureido group, and a cyano group.

20. The kit according to claim 19, wherein the reactive organic group is selected from an epoxy group, a (meth)acryloxy group, an alkenyl group, a mercaptoalkyl group, and an aminoalkyl group.

21. The kit according to claim 1, wherein the concave particles are formed in cross-section from a small internal arc, a large external arc and segments which join together the ends of the respective arcs, the width between the two ends of the small internal arc being in the range from 0.01 to 8 μm on average, the width between the two ends of the large external arc being in the range from 0.05 to 10 μm on average and the height of the large external arc being in the range from 0.015 to 8 μm.

22. The kit according to claim 19, wherein the siliconized concave particles are present in the first composition in a content of 0.1 to 15 wt. % relative to the total weight of the first composition.

23. The kit according to claim 1, wherein the silicone elastomer is a non-emulsifying silicone elastomer.

24. The kit according to claim 23, wherein the non-emulsifying silicone elastomer is present in the first composition in a content of 0.1 to 10 wt. % relative to the total weight of the composition.

25. The kit according to claim 1, wherein the first and/or the second composition further comprises at least one oil.

26. The kit according to claim 25, wherein the first and/or the second composition comprises a volatile silicone oil.

27. The kit according to claim 25, wherein the first and/or the second composition comprises an oil ester of formula R₁COOR₂ in which R₁ represents the residue of a branched fatty acid having from 6 to 10 carbon atoms and R₂ represents a hydrocarbon chain, or a mixture of hydrocarbon chains, containing from 10 to 18 carbon atoms.

28. The kit according to claim 25, wherein the oil is present in the first and/or the second composition in a content in the range from 1 to 80 wt. % relative to the total weight of the individual composition.

29. The kit according to claim 1, wherein the first and/or the second composition further comprise an aqueous phase in a content of 5 to 80 wt. % relative to the total weight of the individual composition.

30. The kit according to claim 29, wherein the first and/or the second composition comprises water at a content in the range from 10 to 75 wt. % relative to the total weight of the composition.

31. The kit according to claim 1, wherein the first and/or second composition of the kit further comprises at least one pulverulent colourant comprising at least one pigment and/or one nacre.

32. The kit according to claim 31, wherein the pigment and/or the nacre is present in the first composition of the kit in a content in the range from 0.01 to 6 wt. % relative to the total weight of the composition.

33. The kit according to claim 1, wherein the first composition does not comprise pigment.

34. The kit according to claim 31, wherein the pigment and/or the nacre is present in the second composition of the kit in a content of 0.01 to 20 wt. %.

35. The kit according to claim 1, wherein the first and/or second composition of the Kit further comprises at least one filler.

36. The kit according to claim 1, wherein the first and/or the second composition of the kit are in the form of a water-in-oil emulsion.

37. The kit according to claim 1, wherein the first and the second composition of the kit are in the form of a water-in-oil emulsion.

38. A method, comprising:
i) forming a first deposit on a keratinic materials;
ii) on all or part of the first deposit, forming a second deposit, said first and second deposits resulting from the application of a first and a second composition of a kit comprising a first and a second composition, the first composition comprising at least one silicone elastomer and siliconized concave particles, the second composition being different from the first and comprising a physiologically acceptable medium.
